(19) United States
(12) Patent Application Publication
(10) Pub. No.: US 2024/0366886 A1
(43) Pub. Date: Nov. 7, 2024

(54) Hypodermic Needle Destruction

(71) Applicant: NSMART TRADING LIMITED, Knowsley (GB)

(72) Inventor: Clifford Kirby, Sefton Village Merseyside (GB)

(21) Appl. No.: 18/577,535

(22) PCT Filed: Jul. 9, 2021
(86) PCT No.: PCT/GB2021/051754

§ 371 Date: Jan. 8, 2024

(51) Int. Cl. A61M 5/32

(52) U.S. Cl. CPC A61M 5/3278; A61M 2005/3283

(57) ABSTRACT

A hypodermic needle destruction device includes a main body housing a power source and a controller; a recess for receiving, in use, a needle destruction module; and means for retaining the needle destruction module at least partially within the recess. The needle destruction module has clamping and tip electrodes that respectively clamp and axially compress a hypodermic needle, and a containment tube that inhibits bowing of the needle. When a current is passed through the needle via the electrodes and an axial force applied, the needle is sterilised and blunted.

### CROSS REFERENCE TO RELATED APPLICATIONS

This application is a U.S. national phase of International Application No. PCT/GB2021/051754, filed Jul. 9, 2021, the contents of which are incorporated by reference herein in their entirety.

### FIELD

This invention relates to hypodermic needle destruction.

### BACKGROUND

The widespread use of hypodermic needles in medical and domestic settings is increasingly widespread. A hypodermic needle is a single-use device, which needs to be disposed of safely if needle stick injuries and cross-infection are to be avoided. Traditionally, a “sharps bin” has been used to store used hypodermic needles and/or syringes until they can be destroyed, for example, by incineration. A drawback of sharps bins subsists in the “waste lifecycle” thereof, as well as the fact that there is also a risk of cross-contamination and/or needlestick injury associated with handling sharps bins.

More recently, there has been a move towards hypodermic needle destruction at the point of use. Various devices have been proposed for this purpose, which include means for rendering the needle sterile and/or blunt to reduce or avoid cross-contamination and needlestick injuries, respectively.

One commercially successful hypodermic needle destruction device is disclosed in our own earlier patent application [EP3024519, Needlesmart Ltd, 1 Jun. 2016] in which a used hypodermic needle is contacted by clamping and tip electrodes and an electric current passed through the needle via the electrodes whilst applying an axial force to soften/melt the needle and simultaneously compress its tip into a blunt ball. The process of heating the needle sterilises it, whilst at the same time, the process of applying an axial compression, deforms the needle into a non-sharp object, which is safe for handling thereafter.

One of the main envisaged user groups of hypodermic needle destruction devices are home users, who need to administer injections regularly. For example, patients suffering from diabetes often have to inject insulin several times per day, and this leads to a large number of hypodermic needles being used in a non-medical setting. Other instances where there can be extensive hypodermic needle use is in the treatment and/or management of drug-addicted patients, or those undertaking palliative care or long-term pain relief.

### SUMMARY

A need therefore exists for a “domestic grade”, user-friendly hypodermic needle destruction device, and preferably one which is portable and easily maintained/serviced.

Aspects of the invention are set forth in the appended independent claim or claims. Preferred and/or optional features are set forth in the appended dependent claims.

According to an aspect of the invention, there is provided a hypodermic needle destruction device comprising a main body housing a power source and a controller, a recess for receiving, in use, a needle destruction module, and means for retaining the needle destruction module at least partially within the recess.

The invention therefore provides a hypodermic needle destruction device that has a removable and/or replaceable needle destruction module, which can render it better suited to a home/non-clinical environment.

The destruction of hypodermic needles using a device such as described in EP3024519 often creates debris, which can accumulate over time within the needle destruction module. Ordinarily, this would necessitate a periodic maintenance procedure, which is acceptable in a healthcare setting, but which is generally unacceptable in a domestic environment. By making the needle destruction module a separate part of the hypodermic needle destruction device, when the needle destruction module begins to lose efficacy, or reaches the end of its serviceable life, it can simply be replaced with a new needle destruction module, but the remainder of the hypodermic needle destruction device can be re-used.

The hypodermic needle destruction device also comprises its own power source, such as a rechargeable battery and/or a supercapacitor. A supercapacitor may be preferred because it can deliver charge much faster than a battery, which makes it ideal for use in the short-duration, high-voltage and/or high-current regime required to heat/soften/melt a metallic needle by Ohmic/resistive heating, as shall be explained below. A supercapacitor may also be preferred because it can accept charge much faster than a battery, which makes it ideal for rapid-charging applications, where the time spent charging needs to be minimised. Supercapacitors may also be preferred because they tolerate many more charge and discharge cycles than rechargeable batteries.

The controller suitably comprises a control circuit and/or a microprocessor, which can be configured to perform a variety of functions. Specifically, the controller is suitably adapted, in use, for controlling the needle destruction module, which may include controlling the power thereto, and/or using a sensor for detecting the presence of a hypodermic needle within the needle destruction module. The controller can thus be configured to go into awake/sleep modes (thereby conserving power) when a hypodermic needle is present or not, respectively. A needle presence sensor may comprise a pressure sensor that is activated when a needle is pushed into the needle destruction module, or a wireless sensor that detects the presence of, for example, an RFID tag, or a (e.g. capacitive) proximity sensor.

Suitably, the needle destruction module comprises a clamping electrode arranged to clamp a hypodermic needle inserted therein at, or near to, the hypodermic needle's hub. The clamping electrode suitably comprises a pair of jaws that move apart to accept the needle but which move together once a needle has been inserted to clamp/trap the needle therebetween. It suitably comprises a containment tube into whose bore the hypodermic needle is receivable. A tip electrode is suitably located within the containment tube and is arranged to slide axially within the containment tube to contact a tip of the needle and apply an axially compressive stress to the needle towards its hub. A power source is suitably provided, which when the clamping and tip electrodes contact the needle, passes an electric current through the needle (between at least a portion of the needle located between clamping and tip electrodes). The current passing through the needle suitably causes resistive/Ohmic heating, which heats, then softens and optionally melts the needle tip. Thus, as the axial stress is applied, the tip electrode axially compresses and blunts the needle tip whilst at the same time, the containment tube inhibits or prevents bowing or breaking-up of the needle under the application of the said compressive stress.

The power source can be a DC power source or an AC power source. The controller preferably comprises a voltage and/or current sensor connected to the clamping and tip electrodes, such that the controller can control the voltage across and/or the current between, clamping and tip electrodes. This means that the current and/or the voltage can be limited and/or ramped and/or caused to follow a V-I profile specific to a certain type of needle.

The tip electrode is suitably driven for axial movement by a motor and a lead screw, the speed and direction of which is suitably controlled by the controller. This can be used to maintain the tip electrode in electrical contact with the needle tip during the heating and/or softening and/or melting thereof; otherwise, as the needle softens, there may be a tendency for the needle tip to recede from the tip electrode, thereby breaking the circuit and halting further heating and/or softening and/or melting of the needle.

The containment tube is suitably manufactured from an electrically insulative material so as to avoid short-circuiting clamping and tip electrodes. The containment tube is suitably manufactured from a high-temperature tolerant material, so as to withstand the high temperatures involved in the needle destruction process. Ceramics or glasses (e.g. quartz) are suitable materials for the containment tube, as could be high-temperature polymers and/or insulated metals (e.g. PTFE-coated steel tubing).

In preferred embodiments of the invention, the containment tube is equipped with a heating device adapted, in use, to pre-heat the containment tube prior to the application of the current and/or the axial compressive stress. It has been found that recently-used needles often contain, or are coated with liquids, which form steam droplets/vapours as the needle is heated during the destruction process. Steam/vapour generation can lead to the needle breaking-up into droplets, rather than being compressed into a blunt ball configuration. However, by pre-heating the containment tube, any such vapour does not tend to condense on the inner surfaces of the containment tube, thereby making the needle destruction procedure more reliable.

In order to facilitate insertion/removal of the needle destruction module from the recess of the main body, the needle destruction module suitably comprises its own a sub-housing, which is at least partially receivable within the recess. The sub-housing is suitably a one-way fit within the recess, so as to minimise or remove the chances of incorrectly attaching it to the main body.

The means for retaining the needle destruction module at least partially within the recess may comprise a latching device. This can be, for example, one or more push-button-operated latching pins, which retract to free the needle destruction module from the recess when pressed, but which spring out when the button is released so as to engage complementary formation of the recess. However, in a preferred embodiment of the invention, the latching device comprises an electronically unlockable latching device, such as an RFID reader located at, or near to, a needle-receiving-opening of the needle destruction module. To unlock the latching device, a dummy needle or administrator key is inserted into the device, which comprises an RFID tag, which is read by the RFID reader. The RFID tag suitably contains an unlock code which is received by the RFID reader and passed to the controller. Upon receipt of an acceptable unlock code, the controller can release the needle destruction module from the recess, for example, by retracting one or more solenoid-actuated locking pins.

Suitably, the hypodermic needle destruction device further comprises power input module and preferably a charge controller for charging the rechargeable battery and/or supercapacitor. The power input module could be a charging jack, but preferably comprises an induction coil that receives power from a complementary charging coil of a charging device or docking station. Wireless charging is preferred as it enables the hypodermic needle destruction device to have a higher IP rating (which is better for portable devices) than might otherwise be the case if a charging jack with open connections were present on, or within, the main body.

In a preferred embodiment of the invention, the controller further comprises a data logging module, which can be configured for logging a variety of data, such as, but without being restricted to:

A unique ID of the hypodermic needle destruction device. This can be tied, for example, in an external database, to an individual person. This means that by monitoring the use of a hypodermic needle destruction device with a particular unique ID, an individual's usage can monitored albeit in an anonymised fashion.

A unique ID of the needle destruction module. This can be, for example, a serial number, and this is useful for estimating the remaining service life of a given needle destruction module. It also permits multiple users to share a hypodermic needle destruction device, but where each user uses their own needle destruction module. This means that by monitoring the use of a particular hypodermic needle destruction device with a particular unique ID in combination with a needle destruction module having a unique ID, an individual's usage can monitored albeit in an anonymised fashion.

A total number of uses of the hypodermic needle destruction device. This could be used to estimate the remaining duty cycle of a rechargeable battery or supercapacitor of the device. It could also be used to monitor for calibration cycles and the like whereby a controller requires recalibration and/or servicing after a certain total number of uses.

A total number of uses of the needle destruction module, which can be used to estimate the remaining life of the needle destruction module. For example, each needle destruction module could be designed to have a duty cycle of, say, 500 uses and a count-down can be used to inform the user when it is approaching, or time to change the needle destruction module and/or to order a replacement.

In certain situations, the needle destruction module may fail, and so recording the number of successful/unsuccessful uses of the needle destruction module can be beneficial for monitoring performance of the device.

The date and/or time of each use of the needle destruction module. This data can be used for cross-checking compliance with a prescribed mediation regimen.

A voltage and/or current profile associated with each use of the needle destruction module. This may be useful for diagnostic purposes, and/or for estimating which types of needle have been destroyed on each use. It can also be used to check for calibration issues and/or wear and tear of the device.

In a preferred embodiment of the invention, the controller also comprises an input-output module for exporting data from the data logging module to an external database. This can be useful for various monitoring purposes, as outlined above. However, one real advantage of exporting data to an external database is the ability then to configure supply chains and/or logistics for a given patient. For example, a particular patient may be given 100 does of insulin in pre-packaged syringes, along with a prescription or guidance on when to inject themselves. The user can then administer their doses, but as their supply of mediation depletes, by interfacing with an external database, a repeat prescription can be ordered, say, for another 100 doses, which can be timed to arrive with the patient just before their previous supply depletes completely. This has the advantage of being able to deliver smaller, albeit more frequent quantities of medication, thereby reducing over-shelf-life wastage, as well as limiting supply (which may be particularly advantageous for drug-addicted patients). The “just-in-time” nature of the data logging, especially when used in conjunction with unique IDs, permits anonymised, controlled and regulated delivery of medications to uniquely-identifiable patients. The data logging/data export functions can also be used to order collection of clinical waste in a timely fashion and other advantages of a real-time or near-time usage monitoring system will be readily appreciated by the skilled reader.

In order to safeguard any personal information, the controller preferably comprises an encryption module for encrypting data stored by the data logging module.

As previously mentioned, a docking station is suitably provided for receiving the main body portion. The docking station may comprise a power output module that is complementary with the power input module of the hypodermic needle destruction device for charging it. Additionally or alternatively, the docking station may comprise an input-output module for receiving data exported from the data logging module. This permits transference of data from the device to the docking station. Suitably, this is complemented by a data checking system, which once confirmed that data has been successfully exported to the docking station, it is permanently deleted from the hypodermic needle destruction device. This further safeguards the hypodermic needle destruction device form cyber-attacks. Additionally or alternatively, the docking station may comprise an input-output module for exporting data to the controller. This can be used for firmware/software updates. Additionally or alternatively, the docking station may comprise an internet or network interface for connecting to an external database, such as the database of a healthcare professional.

### BRIEF DESCRIPTION OF THE DRAWINGS

A preferred embodiment of the invention shall now be described, by way of example only, with reference to the accompanying drawings in which:

FIG. 1 is a schematic, perspective view of a hypodermic needle destruction device in accordance with an embodiment of the invention;

FIG. 2 is a schematic, perspective view of the hypodermic needle destruction device of FIG. 1, albeit with the needle destruction module removed;

FIG. 3 is a perspective view of the needle destruction module shown in FIG. 2;

FIG. 4 is a perspective view of the needle destruction module shown in FIG. 2;

FIG. 5 is a schematic system diagram of an embodiment of a hypodermic needle destruction device; and

FIG. 6 is a schematic system diagram of the hypodermic needle destruction device shown in FIG. 5 during use.

### DETAILED DESCRIPTION

Referring to the drawings, a hypodermic needle destruction device 10 in accordance with the invention comprises a main body 12, which houses its own power source 14, which is suitably a super capacitor for the reasons previously described. The super capacitor 14 is housed within a rear portion of the main body 12 and a front end of the main body 12 has a syringe receiving aperture 16 therein into which the end of a hypodermic syringe/needle assembly can be placed. The hypodermic syringe/needle is inserted needle-first into the aperture 16 until it reaches an end stop (not shown) and this activates the hypodermic needle destruction 10 in order to carry out a destruction procedure. When the syringe/needle is properly inserted, an indicator LED 18 illuminates to signify that fact, and the needle destruction module 20 then goes into operation, as shall be described below.

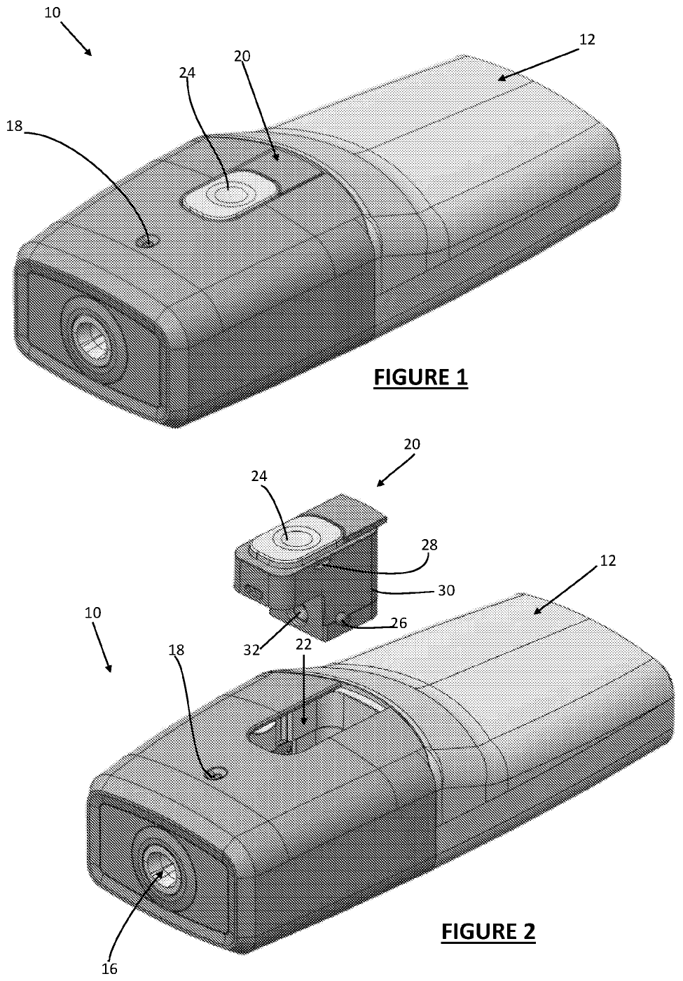

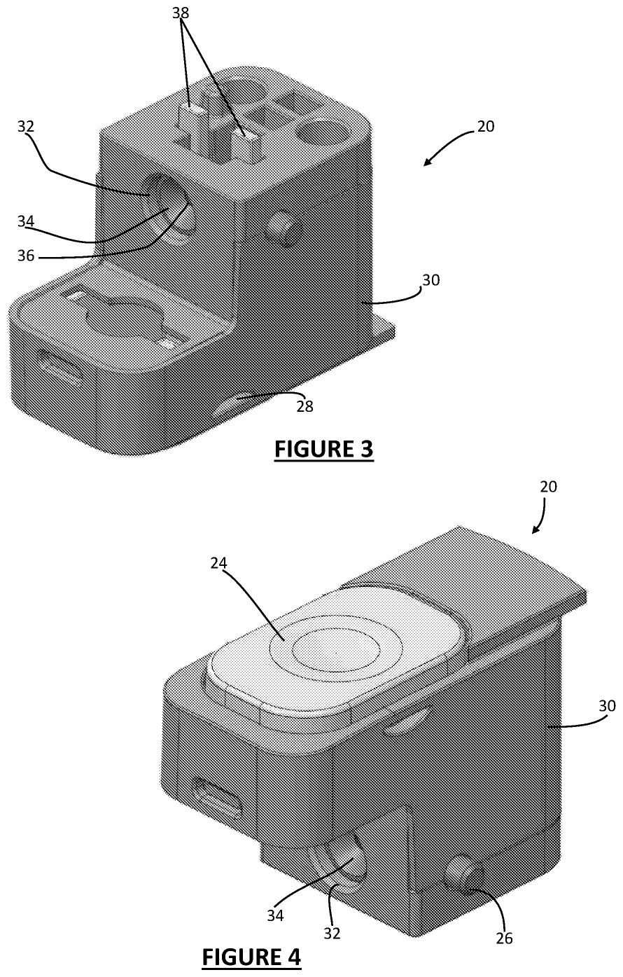

It can be seen, by comparing FIGS. 2 and 3 that the main body 12 comprises a recess 22, that is shaped and sized so as to slidingly receive the needle destruction module 20. When the needle destruction module 20 is fully-inserted, as shown in FIG. 1, its upper surface lies flush with the surface of the main body 12. A release button 24 is provided, which mechanically connects to a latching pin 26 on either side of the needle destruction module. By depressing the release button 24, the pins 26 are retracted, and the needle destruction module 20 can be extracted, for example, by using a tool or finger nails in a recess 22 provided on the needle destruction module 20.

As can be seen from FIG. 2 of the drawings, the overall shape of the housing 30 of the needle destruction module 20 is asymmetric, which means that it can only be inserted into the recess 22 in one, namely, correct orientation.

As can be seen clearly from FIG. 2 of the drawings, the needle destruction module 20 has an opening 32 at one end thereof, which receives a hypodermic needle as shall be explained herein below.

FIGS. 3 and 4 of the drawings show the needle destruction module 20 in close-up, and it can be seen that the needle receiving aperture 32 has a centraliser 34, which guides the needle towards the longitudinal axis of a bore 36 of the device. A set of clamping electrodes 38, move towards each other when a needle has been inserted, and these form a clamping electrical contact with the needle at, or near to its hub.

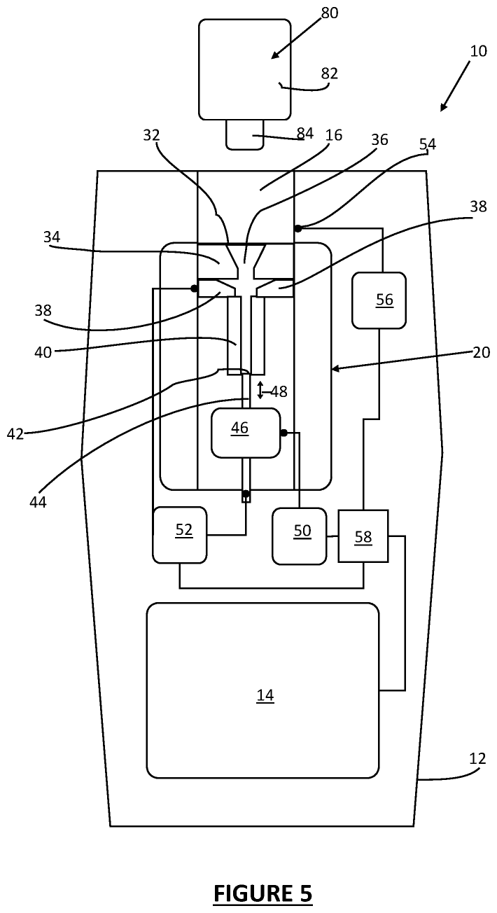

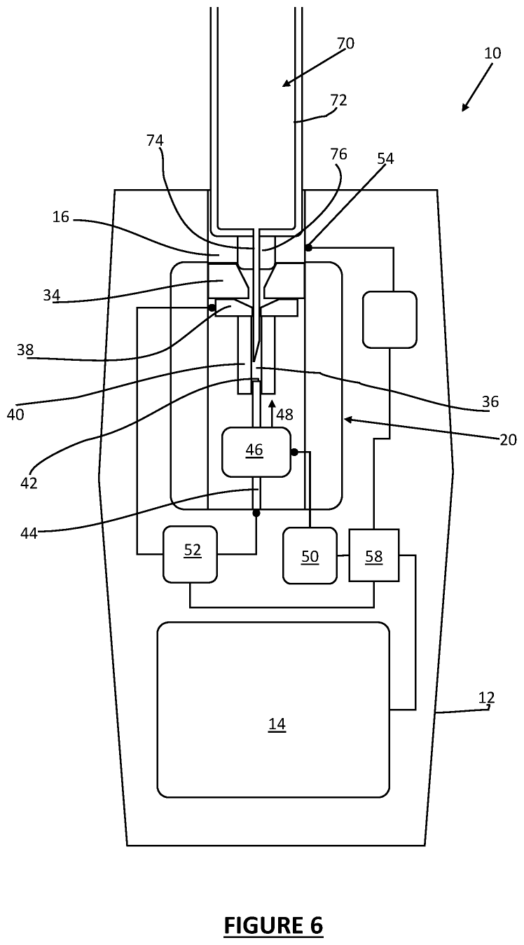

Referring now to FIGS. 5 and 6 of the drawings, it can be seen how the needle destruction module 20 fits into the housing 12 of the overall hypodermic needle destruction device. It can also be seen how the syringe receiving aperture 16 is coaxial with the needle receiving aperture 32 of the needle destruction device 20, as well as the centraliser 34 and the bore 36. The clamping electrodes 38 are also centralised on the bore 36, and they lead into a tubular containment tube 40 located within the needle destruction module 20, which is also coaxial with the bore 36.

At the opposite end of the opening 32 within the bore 36 is a tip electrode 42, which is formed at the end of a lead screw 44, which is in turn driven by a motor 46. Rotation of the rotor 46 advances or receives the lead screw 44, as indicated by the arrow 48 in the drawings, and this moves the tip electrode 42 towards, or away from the opening 32 as the case may be.

The motor 46 is controlled by a motor driver 50, which draws its power from the super capacitor 14 within the main body. The motor driver is configured to control the speed and/or direction of the motor's movement, and hence the movement 48 of the tip electrode 42 within the containment tube 40.

Meanwhile, the clamping 38 and tip 42 electrodes are connected to a power supply 52, which also draws its power from the super capacitor 14. A voltage can thus be applied between the clamping 38 and tip 42 electrodes during use of the device 10. The voltage controller 52 is able to regulate the voltage and/or current at the clamping and tip electrodes, and hence the current passing through the needle, during use. The voltage may be an AC or a DC voltage, it may be ramped or follow some other profile, which is optimised for destroying needles of a particular type.

As previously mentioned, the insertion or removal of a syringe into the device 10 is detected using a sensor 54, which is connected to a sensing module 56.

A central processor 58 is provided, which controls the operation of the motor controller 50, the voltage driver 52 and the sensor controller 52, and the main processor 58 also draws its power from the super capacitor 14. Not shown in FIGS. 5 and 6, for clarity only, are other ancillary components, such as a charging circuit, a charging jack, a power on/off switch, etc.

In use, as shown in FIG. 6 of the drawings, a syringe assembly 70 is inserted into the receiving aperture 16 of the device 10. The syringe assembly comprises a syringe body 72, and a hypodermic needle 74 affixed thereto via a hub 76. The hub may be a screw or bayonet fit device, or the needle 74 could be moulded onto the syringe body 72 via the hub 76.

Upon insertion of the syringe assembly 70 into the receiving aperture 16, the presence of the syringe assembly 70 is detected by the sensor 54. It could also be detected by an axial force applied to the centraliser 34, and various other ways of detecting the presence or absence of a syringe assembly in the device 10 are easily envisaged.

The centraliser 34 guides the tip of the needle towards the axis of the assembly and thus centralises it within the clamping electrodes 38 and, eventually, the containment tube 40. Once inserted, the containment tube 40 begins a pre-heat process, whereby its temperature is elevated so as to avoid forming condensation inside it during the subsequent needle destruction process.

As can be seen by comparing FIG. 5 and FIG. 6, once the needle 74 has been inserted into the bore 36, the clamping electrodes 38 move together so as to clamp the needle 74 and form an electrical contact therewith. The motor 46 is then driven so as to advance 48 the tip electrode 42 towards the tip of the needle 74. The voltage controller 52 then applies voltage between the clamping 38 and tip 42 electrodes and when the tip electrode 42 eventually contacts the tip of the needle 74, an electric current passes through the needle 74 between the clamping electrode 38 and the tip electrode 42. Resistive/ohmic heating then occurs, which causes the needle 74 to heat, and then soften, and it may ultimately melt. At the same time, the motor 46 continues to drive the lead screw 44 so that the tip electrode 42 applies an axial compressive stress to the needle 74. The combination of heating/softening/melting with an axial force applied to the tip of the needle 74 causes the needle to be blunted and compressed. The containment tube 40 constrains the needle 74 to prevent it from bowing or breaking up during the destruction process, and this results in the needle being blunted and heated to above a sterilisation temperature simultaneously. This procedure effectively renders the needle 74 safe as it has been both sterilised and blunted in one operation.

Once this has been accomplished, the motor 46 reverses direction, thereby retracting the tip electrode, and the clamping electrodes 38 are released. The indicator LED 18 on the outer housing 12 of the device 10 can then change colour or extinguish to indicate that the needle destruction process has been completed successfully.

As can be seen in FIG. 5 of the drawings, an unlocking key 80 is provided, which has a main body 82 and a tip portion 84 that somewhat resembles the shape/configuration of a hub 76 of a hypodermic needle assembly 70. When the unlocking key 80 is inserted into the receiving aperture 16, its presence is detected by the sensor 54. However, the tip portion 84 comprises an RFID tag, which contains an unlocking code. A complimentary RFID reader built into the device 10, for example at position 54, reads the RFID tag and determines whether or not it is an authenticated unlock code. If so, the processor 58 retracts a set of solenoids, which retract the locking pins 26 previously described and permit the needle destruction module 20 to be removed.

Not explicitly shown in the drawings is an I/O module and a database module forming part of the main processor 58. The data logging module records all events associated with the hypodermic needle destruction device, as well as process parameter, and stores them in an encrypted fashion in an on-board memory (not shown). The idea behind this is that the device 10 logs each and every use of the hypodermic needle destruction device and whilst doing so, it records the date/time, the unique ID of the hypodermic needle destruction device 10, the unique ID of the needle destruction module 20, as well as the process parameters used by the voltage controller 52 and the motor controller 50. This data is used to populate an internal database, which stores all of the usage statistics and process parameters for the hypodermic needle destruction device 10.

The processing module 58 also comprises an I/O module, which enables data to be exported to an external database, preferably in an encrypted fashion. This permits all of the logged data to be exported to an external database for subsequent review/analysis. This can be used for monitoring the use of the device, the correct functioning of the device, as well as for ordering fresh supplies to the uniquely identifiable user, as well as ordering waste collection services, etc.

The invention is not restricted to the details of the foregoing embodiments, which are merely exemplary of the invention.

1-23. (canceled)

24. A hypodermic needle destruction device comprising a main body housing a power source and a controller, the main body having a recess for receiving, in use, a sub-housing containing a needle destruction module, and latching device for releasably retaining the sub-housing, and hence the needle destruction module, at least partially within the recess.

25. The hypodermic needle destruction device of claim 24, wherein the controller comprises a control circuit and/or a microprocessor adapted, in use, to control the needle destruction module.

26. The hypodermic needle destruction device of any claim 24, further comprising a sensor for detecting the presence of a hypodermic needle within the needle destruction module.

27. The hypodermic needle destruction device of claim 24, wherein the needle destruction module comprises:
a clamping electrode arranged to clamp a hypodermic needle inserted therein at, or near to, the hypodermic needle's hub;
a containment tube into whose bore the hypodermic needle is receivable;
a tip electrode located within the containment tube and being arranged to slide axially therewithin to contact a tip of the needle and apply an axially compressive stress to the needle towards its hub; and
a power module adapted, when the clamping and tip electrodes contact the needle, to pass an electric current through the needle thereby heating and/or softening and/or melting the needle such that the said axial stress axially compresses and blunts the needle whilst the containment tube inhibits or prevents bowing or breaking-up of the needle under the application of the said compressive stress.

28. The hypodermic needle destruction device of claim 27, wherein the power module is a DC power source.

29. The hypodermic needle destruction device of claim 27, wherein the power module is an AC power source.

30. The hypodermic needle destruction device of claim 27, wherein the controller comprises a voltage and/or current sensor connected to the clamping and tip electrodes, and wherein the controller is adapted, in use, to control the voltage across and/or the current between, clamping and tip electrodes.

31. The hypodermic needle destruction device of claim 27, wherein the tip electrode is driven for axial movement by a motor and lead screw, the speed and direction of the motor being controlled by the controller so as to maintain the tip electrode in electrical contact with the needle tip during the heating and/or softening and/or melting thereof.

32. The hypodermic needle destruction device of claim 27, wherein the containment tube is equipped with a heating device adapted, in use, to pre-heat the containment tube prior to the application of the current and/or the axial compressive stress.

33. The hypodermic needle destruction device of claim 24, wherein the latching device comprises a push-button operated latching device.

34. The hypodermic needle destruction device of claim 24, wherein the latching device comprises an electronically unlockable latching device.

35. The hypodermic needle destruction device of claim 34, wherein the electronically unlockable latching device comprises an RFID reader adapted, in use, to receive an unlocking code from an RFID tag of an unlocking key.

36. The hypodermic needle destruction device of claim 24, wherein the power source comprises a rechargeable battery.

37. The hypodermic needle destruction device of claim 24, wherein the power source comprises a supercapacitor.

38. The hypodermic needle destruction device of claim 24, further comprising power input module and a charge controller for charging the rechargeable battery of claim 13, or the supercapacitor of claim 14.

39. The hypodermic needle destruction device of claim 28, wherein the power input module comprises an induction coil.

40. The hypodermic needle destruction device of claim 24, wherein the controller further comprises a data logging module for logging any one or more of the group comprising:
a unique ID of the hypodermic needle destruction device;
a unique ID of the needle destruction module;
a total number of uses of the hypodermic needle destruction device; a total number of uses of the needle destruction module;
a number of successful uses of the needle destruction module; a number of unsuccessful uses of the needle destruction module;
the date of each use of the needle destruction module;
the time of each use of the needle destruction module;
a current profile associated with each use of the needle destruction module; and
a voltage profile associated with each use of the needle destruction module.

41. The hypodermic needle destruction device of claim 40, wherein the controller further comprises an input-output module for exporting data from the data logging module to an external database.

42. The hypodermic needle destruction device of claim 40, wherein the controller further comprises an encryption module for encrypting data stored by the data logging module.

43. The hypodermic needle destruction device of claim 24, further comprising a docking station for receiving the main body portion, the docking station comprising any one or more of:
a power output module that is complementary with the power input module of the hypodermic needle destruction device;
an input-output module for receiving exported data from the data logging module;
an input-output module for exporting data to the controller; and
an internet or network interface for connecting to an external database.
